(19) United States
(12) Patent Application Publication
(10) Pub. No.: US 2006/0234386 A1
(43) Pub. Date: Oct. 19, 2006

(54) Method and system for measuring lactate levels in vivo

(75) Inventors: David H. Burns, Montreal (CA); Denis Lafrance, Montreal (CA); Larry Lands, Montreal (CA)

Correspondence Address: BERESKIN AND PARR, 40 KING STREET WEST, BOX 401, TORONTO, ON, M5H 3Y2, CA

(73) Assignee: McGILL UNIVERSITY, Montreal (CA)

(21) Appl. No.: 10/554,698

(22) PCT Filed: Apr. 30, 2004
(86) PCT No.: PCT/IB04/01317

§ 371 Date: Oct. 27, 2005

Related U.S. Application Data
(60) Provisional application No. 60/466,462, filed on Apr. 30, 2003.

(51) Int. Cl. G01N 33/00; A61B 5/00; A61B 5/02

(52) U.S. Cl. 436/94; 600/504; 600/310

(57) ABSTRACT

There is described a system and method for the in vivo determination of lactate levels in blood using Near-Infrared Spectroscopy (NIRS) and/or Near-infrared Raman Spectroscopy (NIR-RAMAN). The method teaches measuring lactate in vivo comprising: optically coupling a body part with a light source and a light detector the body part having tissues comprising blood vessels; injecting near-infrared (NIR) light at one or a plurality of wavelengths in the body part; detecting, as a function of blood volume variations in the body part, light exiting the body part at at least the plurality of wavelengths to generate an optical signal; and processing the optical signal as a function of the blood volume variations to obtain a lactate level in blood.

### CROSS-REFERENCE TO RELATED APPLICATION

This application claims priority of U.S. provisional application No. 60/466,462, filed Apr. 30, 2003. The contents of the references cited throughout the disclosure are incorporated herein by reference.

### TECHNICAL FIELD

The invention relates to the measurement of blood metabolites. More particularly the invention relates to the measurement of lactate using Near-infrared (NIR) spectroscopy.

### BACKGROUND OF THE INVENTION

In critical care, the continuous monitoring of blood lactate is of significant importance. An increase in lactate level reflects an imbalance between lactate production and elimination. Lactate can then be used as a marker for the assessment of tissue perfusion and oxidative capacity. While a whole blood lactate concentration of less than 2 mmol/L is considered as normal (Mizock B. A. et al., Crit. Care Med. 20: 80-93, 1992), concentrations higher than 4 mmol/L have been found in association with myocardial infarction (R. J. et al., Circ. Shock 9: 307-315, 1982), cardiac arrest (Weil M. H. et al., Crit. Care Med. 13: 888-892, 1985), circulatory failure (Broder G. et al., Science 143: 1457-1459, 1964; Weil M. H. et al., Circulation 41: 989-1001, 1970) and in emergency trauma situations (Aduen J. et al., JAMA 272: 1678-1685, 1994, 44). Likewise, the change in pattern or the trend towards an increase of blood lactate is a good indicator of survival (Cowan B. N. et al., Anaesthesia 39: 750-755, 1984; Vincent J. L. et al., Crit. Care Med. 11:449-451, 1983). In all these cases, measurements of lactate levels are of prognostic significance and have to be performed by a rapid and robust method.

However, most of the standard clinical methods for lactate analysis are not adapted for continuous lactate monitoring (Baker D. A. et al, Anal. Chem. 67: 1536-1540, 1995; Soutter W. P. et al., Br. J. Anaesth. 50: 445-450, 1978; Williams D. L. et al., Anal. Chem. 42; 118-121, 1970). They often require substantial sample preparations and for this reason, do not offer the possibility to the clinician of concurrent in vivo or ex vivo monitoring of lactate level in a continuous manner. To achieve at patient monitoring of lactate, several in vivo biosensors, Baker D. A. et al, Anal. Chem. 67: 1536-1540, 1995; Pfeiffer D. et al., Biosens. Bioelectron. 12: 539-550, 1997; Wang D. L. et al., Anal. Chem. 65: 1069-1073, 1993; Yang Q. et al., Biosens. Bioelectron. 14: 203-210,1999; ex vivo, Gfrerer R. J. et al., Biosens. Bioelectron. 13: 1271-1278, 1998; Kyröläinen M. et al., Biosens. Bioelectron. 12: 1073-1081, 1997; Meyerhoff C. et al., Biosens Bioelectron. 8: 409-414, 1993; and microdialysis procedures Dempsey E. et al., Anal. Chim. Acta 346: 341-349, 1997; Kaptein W. A. et al., Anal. Chem. 70: 4696-4700, 1998; have been developed. Although they overcome some of the problems, these methods suffer from several drawbacks. Biofouling, biocompatibility, thrombi formation, calculation of the recovery and discomfort for the subjects are some of the major disadvantages and problems of these techniques that ultimately remain invasive devices (Ash S. R. et al., ASAIO J. 38: M416-M420, 1992, Johne B. et al., J. Immunol. Methods 183: 167-174, 1995; Justice J. B., Jr., J. Neurosci. Methods 48: 263-276, 1993; Reach G. et al., Anal. Chem. 64: 381A-386, 1992).

Previous studies have shown the potential of near infrared spectroscopy (NIRS) to monitor non-invasively tissue oxygenation, Boushel R. et al., Acta Physiol. Scand. 168: 615-622, 2000; Iwai H. et al., Ther. Res. 21:1560-1564, 2000; Oda M. et al., Reza Kenkyu 25: 204-207, 1997; Thorniley M. S. et al., Biochem. Soc. Trans. 16: 978-979, 1988; Thorniley M. S. et al., Biochem. Soc. Trans. 17:903-904, 1989; and Wang F. et al., Ziran Kexueban 39: 16-19, 1999; and other metabolites, Arnold M. A., Curr. Opin. Biotechnol. 7: 46-49, 1996; Heise H. M. et al., Artif. Organs 18: 439-447, 1994; Heise H. M., Horm. Metab. Res. 28: 527-534, 1996; Heise H. M. et al., AIP Conf Proc. 430: 282-285, 1998; Heise M. et al., J. Near Infrared Spectrosc. 6: 349-359, 1998; Marbach R. M. et al., Appl. Spectrosc. 47: 875-881, 1993; Mueller U. A. et al., Int. J. Artif. Organs 20: 285-290, 1997; and Robinson M. R. et al., Clin. Chem. 38:1618-1622, 1992. Likewise recently, in vitro measurement of lactate was also made using Near Infrared Spectroscopy Lafrance D. et al., Appl. Spectrosc. 54: 300-304, 2000; Lafrance D. et al., Can. J. Anal. Sci. & Spectrosc. 45: 34-38, 2000.; Lafrance D. et al., Talanta (To be published).

In view of the above there is clearly a need for more effective measurement methods for blood metabolites.

### SUMMARY OF THE INVENTION

The present invention provides a system and method for the in vivo determination of lactate levels in blood using Near-Infrared Spectroscopy (NIRS)and/or Near-infrared Raman Spectroscopy (NIR-RAMAN).

In one embodiment of the method, a part of the body is optically coupled with a near infrared light source and detector. Light is injected and detected at multiple wavelengths to produce an optical signal that can be processed to derive levels of blood metabolites such as lactate. The method enables measurements of lactate to be performed more rapidly than existing methods and to allow continuous monitoring. Furthermore, when the processor is coupled to a monitor, signals perceptible to a user may be generated to indicate lactate levels differing from predetermined levels. These advantages can be exploited in clinical situations or during physiological exercises studies for example.

In a further aspect of the method NIRS may be used to measure lactate levels in blood samples using transmission or reflectance spectroscopy.

In yet a further embodiment there is provided a system for the in vivo measurement of lactate comprising an NIR light source, means for optically coupling the source to a body part and means for optically coupling the body part to a detector, means to process the diffuse reflectance optical signal to generate a measure of lactate levels and monitoring means to compare measured lactate levels to predetermined levels and to trigger signals perceivable by a user when the compared levels are within a predetermined range.

### BRIEF DESCRIPTION OF THE DRAWINGS

Further features and advantages of the present invention will become apparent from the following detailed description, taken in combination with the appended drawings, in which:

FIG. 1 is an example of a correlation coefficient plot based on diffuse reflectance spectra from the fingernails of each of the subjects tested;

FIG. 2 is an example of a 2D-NIR correlation spectra (synchronous and asynchronous) based on diffuse reflectance spectra from the fingernails of each of the subjects tested;

FIG. 3 is an example of a PRESS plot for lactate cross-validation model based on the 1500 to 1750 nm spectral range;

FIG. 4 is an example of a calibration coefficient plot using 4 PLS factors for the in vivo determination of lactate;

FIG. 5 is an example of NIRS estimated vs. Kodak Vitros values for in vivo lactate measurements for each of the ten subjects (Cross-validation model: 4 PLS factors based on 1500-1750 nm spectral segment; n=40, R²=0.74, RMSCV=2.21 using a leave-4-out cross-validation procedure);

FIG. 6 is an example of NIRS estimated vs. lactate referenced values for in vivo lactate measurements for each of the ten subjects (Cross-validation model: 5 PLS factors based on 1500-1750 nm spectral segment; n=30, R²=0.97, RMSCV=0.76 mmol/L using a leave-4-out cross-validation procedure); and

FIG. 7 is a schematic representation of an embodiment of the system of the present invention.

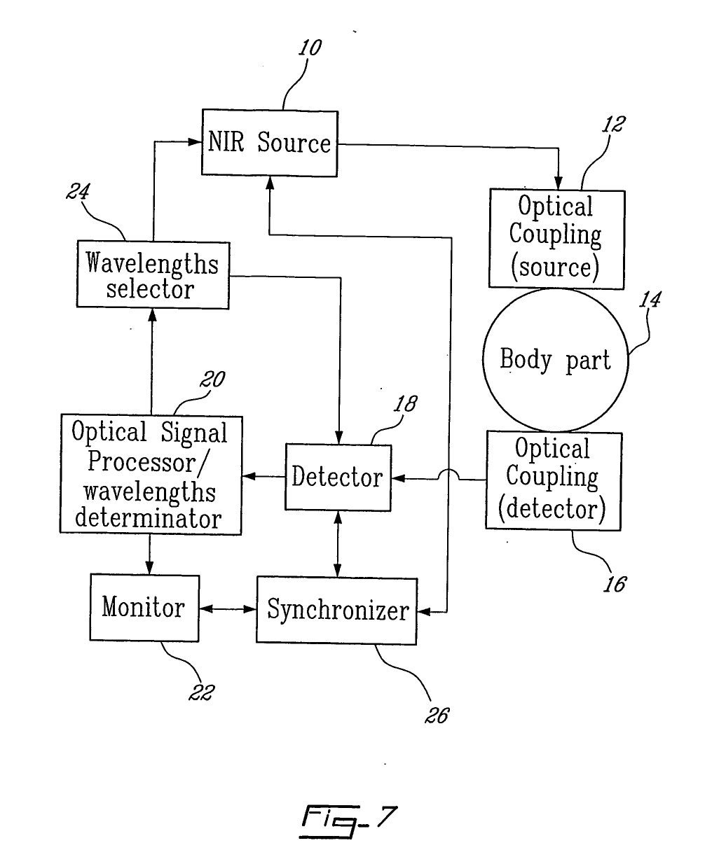

It will be noted that throughout the appended drawings, like features are identified by like reference numerals.

### DETAILED DESCRIPTION OF THE PREFERRED EMBODIMENT

In one embodiment of the present invention there is provided a method and system for the in vivo measurement of blood lactate levels using NIR reflectance spectroscopy. The method involves the optical coupling of a body part with a NIR source and a suitable detector for measuring light exiting from the body part. By analyzing the light exiting the body at predetermined wavelengths, the method enables the in vivo measurement of blood lactate levels. The selection of the appropriate wavelengths will be further described below. The non-invasive nature of the method permits frequent measurements of blood lactate to be made in a continuous manner. Furthermore, by linking the lactate results output with a monitor device, the system and method provides a means for triggering an alarm in response to changes in blood lactate levels. Abnormal levels may occur in individuals suffering myocardial infarction, cardiac arrest, circulatory failure, emergency trauma and the like or during exercises. The alarm enables one to decide whether corrective measures should be taken.

While several parts of the body may be suitable for the acquisition of data, digits (fingers and toes) are preferred. More preferably the nail portion of digits is used since the nail is relatively transparent to NIR and the nail bed is rich in capillary blood vessels.

To determine the predominant change in the spectra, 2D correlation spectroscopy was used (Noda I., Bull. Am. Phys. Soc. 31: 520-552, 1986; Noda I., J. Am. Chem Soc. 111: 8116-8118, 1989). The technique of 2D correlation spectroscopy was developed for characterizing differences in spectral responses between elements of a set of spectra with certain variations present among them. Two pre-processing steps were used on the spectra before plotting the 2D correlation spectrum. First, all spectra were mean-centered. Mean-centering emphasized the subtle variations in the spectra due to changing species concentrations. To enhance the spectral variations of species over the background and minimize baseline variation, the second derivative of all blood sample spectra was calculated using discrete differences (Holler F. et al., Appl. Spectrosc. 43: 877-882, 1989).

Following the determination of the predominant species in the spectra, Partial Least Squares (PLS) regression analysis was made on the pre-processed data. The PLS method and the second derivative routine have been developed previously and details of the algorithm have been discussed (Arakaki L. S. L. et al., Appl. Spectrosc. 50: 697-707, 1996; Holler F. et al., Appl. Spectrosc. 43: 877-882, 1989). For robust estimation using PLS, a cross validation method was used with 40 unique samples. In this study, blocks of four samples from the same volunteer were left out. Since each individual is excluded and estimated by the nine others, the leave-one-individual-out cross-validation approach ensures that variations between patients could be determined. The final model is developed using ten individual calibration coefficient vectors to estimate the concentration in each of the samples. The prediction error sum of squares (PRESS) with F-test significance comparisons was used to determine the minimum number of statistically significant factors (D.M. et al., Anal. Chem. 60: 1193-1202, 1988). The number of latent variables used in the model presented in this study is determined using the cumulative PRESS calculated from the sum of the ten leave-one-individual-out cross validations. In one embodiment, all programs for input of spectral data, pre-processing, 2D correlation plot and cross-validation were written in Matlab (The Mathworks Inc., South Natick, Mass.). However it will be appreciated that other software may be used which use multilinear regression to develop a calibration vector for lactate.

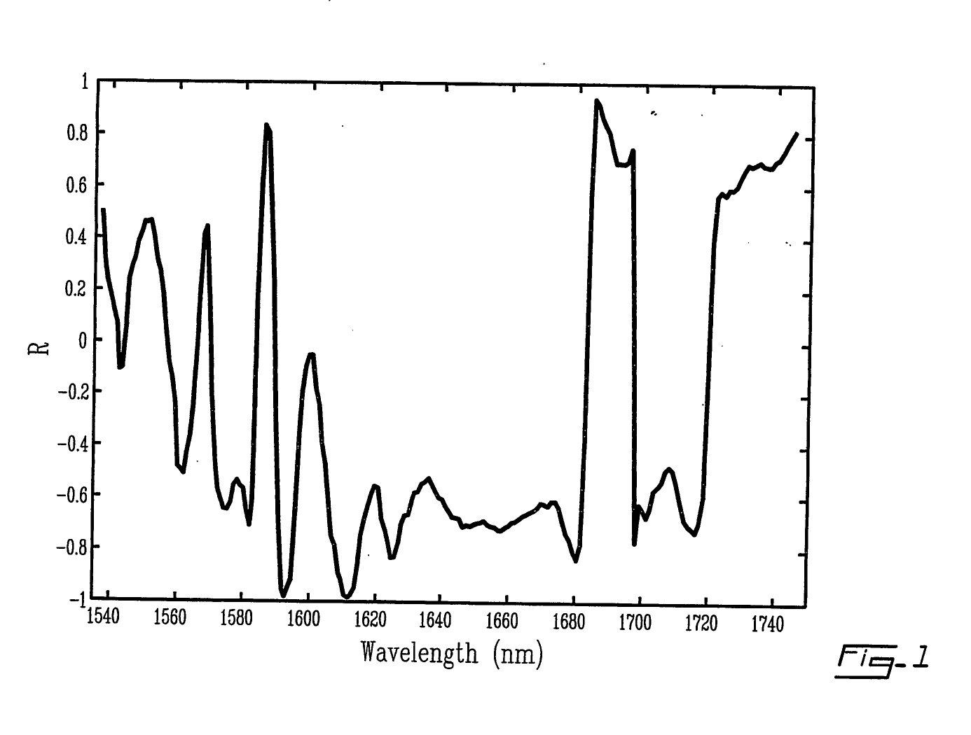

In order to determine wavelengths that mainly correlate over time with spectral changes, a correlation coefficient plot is shown in FIG. 1. Although it was not possible to assign some of the most correlated wavelengths with a particular species (1586 nm, 1593 nm, 1626 nm and 1716 nm), other correlated wavelengths can be assigned to glucose (1612 nm and 1689 nm) and lactate (1675 nm, 1690 nm and 1730 nm). No correlated wavelengths are related to water.

Table I shows changes over time of lactate and glucose concentration for each of ten individuals tested at various time before and after exercise.

TABLE I
Lactate and glucose concentration changes over the course for each of ten individuals.

| | Lactate | | | | Glucose | | | |
|---|---|---|---|---|---|---|---|---|
| | At | | (mmol/L) | | At | | (mmol/L) | |
| | rest | t = 0 min | t = 5 min | t = 10 min | rest | t = 0 min | t = 5 min | t = 10 min |
| Subject 1 | 0.9 | 1.8 | 11.2 | 10.9 | 5.0 | 5.4 | 5.4 | 5.3 |
| Subject 2 | 0.7 | 2.1 | 5.1 | 5.4 | 4.6 | 4.6 | 4.6 | 4.5 |
| Subject 3 | 0.8 | 1.2 | 6.3 | 6.9 | 4.6 | 4.5 | 4.7 | 4.7 |
| Subject 4 | 0.9 | 1.6 | 6.0 | 5.6 | 4.6 | 4.8 | 5.0 | 4.9 |
| Subject 5 | 1.0 | 2.0 | 8.0 | 8.3 | 5.1 | 5.5 | 5.1 | 5.2 |
| Subject 6 | 0.9 | 1.6 | 4.8 | 4.9 | 5.2 | 5.2 | 5.3 | 5.3 |
| Subject 7 | 1.0 | 2.2 | 3.1 | 3.2 | 4.5 | 4.4 | 4.6 | 4.6 |
| Subject 8 | 1.5 | 1.7 | 5.8 | 5.7 | 5.9 | 5.7 | 5.7 | 5.6 |
| Subject 9 | 1.0 | 1.4 | 5.2 | 4.6 | 4.9 | 4.9 | 4.7 | 4.7 |
| Subject 10 | 1.1 | 1.0 | 10.1 | 10.1 | 4.9 | 4.7 | 5.4 | 5.1 |

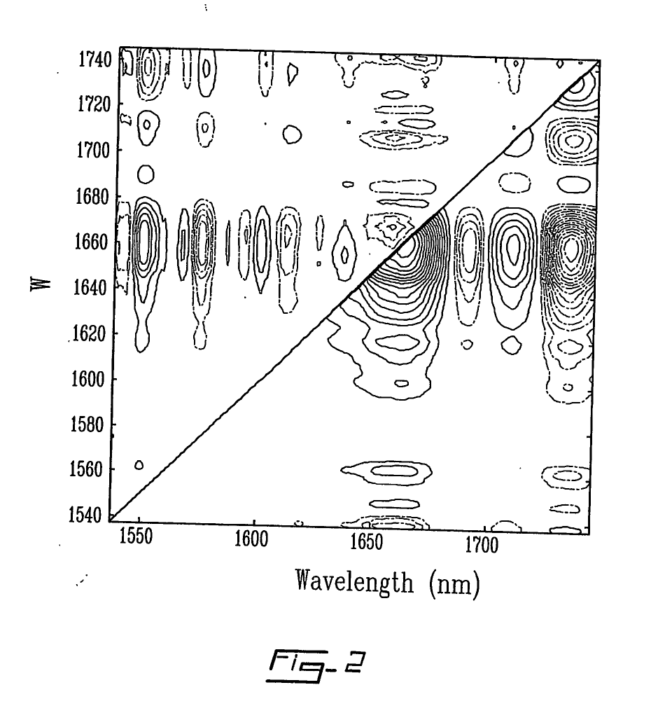

To better understand what induced spectral changes over the course of time, 2D correlation analysis was used. FIG. 2 shows the synchronous (bottom) and asynchronous (top) 2D correlation spectra from human nails bed. The synchronous spectrum represents the simultaneous or coincidental changes of spectral intensity variations measured at two different wavelengths during the 10 minutes interval chosen for the experiment. The synchronous spectrum shows correlation peaks appearing at both on and off diagonal. The on-diagonal peaks or “autopeaks” correspond to the autocorrelation of a wavelength. Thus, the evaluation of the synchronous spectrum along its diagonal provides the overall extent of dynamic fluctuations in the spectral intensity. Likewise, the off-diagonal peaks or “cross-peaks” show the simultaneous changes of signals that occur at two different wavelengths. The magnitude and position of cross-peaks can then be useful to determine whether simultaneous spectral changes in two wavelength regions are coupled (Noda I., Bull. Am. Phys. Soc. 31: 520-552, 1986; Noda I., J. Am. Chem Soc. 111: 8116-8118, 1989; Noda I. et al., Appl. Spectrosc. 54: 236A-248A, 2000). The synchronous spectrum in FIG. 2 shows that the predominant change is centered at 1662 nm, but the peak is broad: In an attempt to assign some of the features to species of interest, standard buffered solutions were prepared. It was determined that in the selected spectral range (1500-1750 nm) lactate shows absorption at 1675, 1690 and 1730 nm, while glucose shows at 1613, 1689 and 1732 nm (Burmeister J. J. et al., Clin. Chem. 45: 1621-1627, 1999). The feature at 1662 appears to be a combination of absorption from fingernail (1660 nm) and lactate (1675 nm). Furthermore, simultaneous changes also appear at 1710 nm and, but with opposite sign, at 1690 nm and 1735 nm. While the feature at 1690 nm can be assigned to lactate, the feature at 1735 appears to be a combination of absorption from lactate (1730 nm), glucose (1732 nm) and fingernail (1740 nm).

The top part of FIG. 2 shows the asynchronous spectrum. The asynchronous spectrum represents the sequential or successive information changes in spectral intensities measured at two different wavelengths (Noda I. et al., Appl. Spectrosc. 54: 236A-248A, 2000). Unlike the synchronous spectrum, the asynchronous plot does not have autopeaks, but only off-diagonal cross-peaks and is antisymmetric with respect to the central diagonal. Furthermore, the sign of the cross-peak can be used to determine the sequential order of the spectral changes that occur. A positive asynchronous cross-peaks at (λ₁, λ₂) indicates that a change at λ₁ occurred predominately before λ₂ in the sequential order of changes. In FIG. 2, out-of phase changes appear at 1636 nm, 1600 nm and 1550 nm and, but with opposite sign, at 1610 nm and 1575 nm. While the small out-of-phase feature at 1610 nm can be assigned to glucose, the other features of the asynchronous spectrum have not been assigned, but can be related to other species of human tissues such as proteins.

In one aspect of the invention, 2D correlation spectroscopy technique led to the identification of two potential species, lactate and glucose that could be monitored through NIR fingernail diffuse reflectance. To confirm which one of lactate or glucose offers the best potential for estimating concentration levels of the metabolite PLS models were determined for both species. However, to develop an acceptable PLS model, no covariance between the multiple components of the sample matrix should be seen. Table II lists the correlation coefficients between measured lactate, glucose and the other parameters.

TABLE II
Correlation coefficients (R) calculated between
lactate and other measured parameters.

| | Glucose | Hematocrit | Temp.-finger | Temp. Mouth |
|---|---|---|---|---|
| Lactate | 0.2668 | 0.3793 | 0.5212 | 0.2441 |
| Glucose | | −0.0633 | 0.0849 | 0.0181 |
| Hematocrit | | | 0.2956 | −0.1742 |
| Temp.-finger | | | | 0.3425 |

No significant correlation was found between these parameters. Likewise, it has previously been shown that variable light scattering from red blood cells can be correlated with pH changes in the samples (Alam M. K. et al., Appl. Spectrosc. 53: 316-324, 1999). The correlation with pH is caused by variations in light scatter due to red blood cells shrinking and swelling as a function of pH (Alam M. K. et al., Appl. Spectrosc. 53: 316-324, 1999). However, such correlation is usually seen in experiments where pH variation is much larger (>1 pH unit) than in a physiological study Lafrance D. et al., Appl. Spectrosc. 54: 300-304, 2000. Furthermore, previous study has shown no correlation between spectral changes and pH variation in samples during a similar protocol to this study, Lafrance D. et al., Appl. Spectrosc. 54: 300-304, 2000.

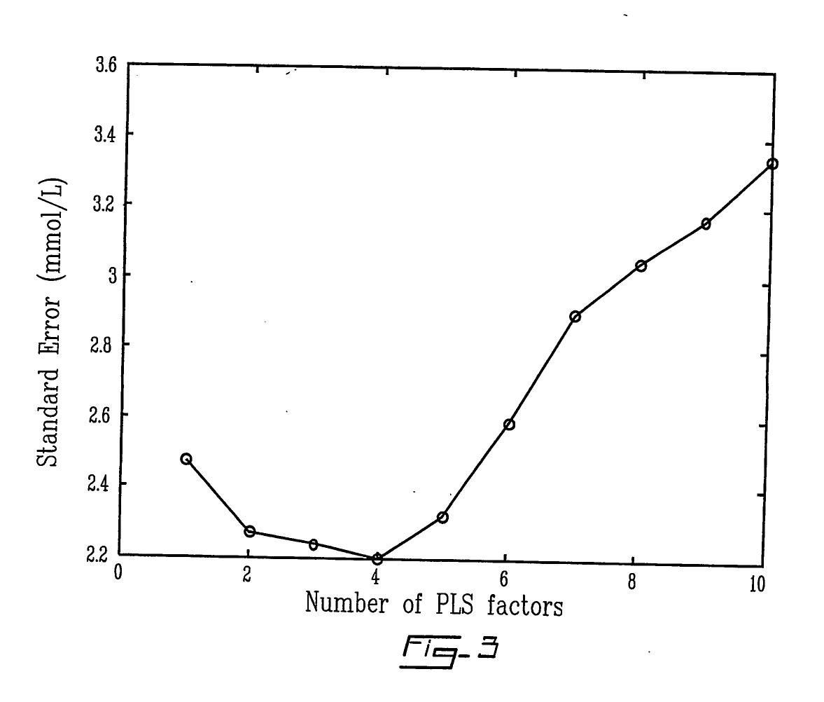

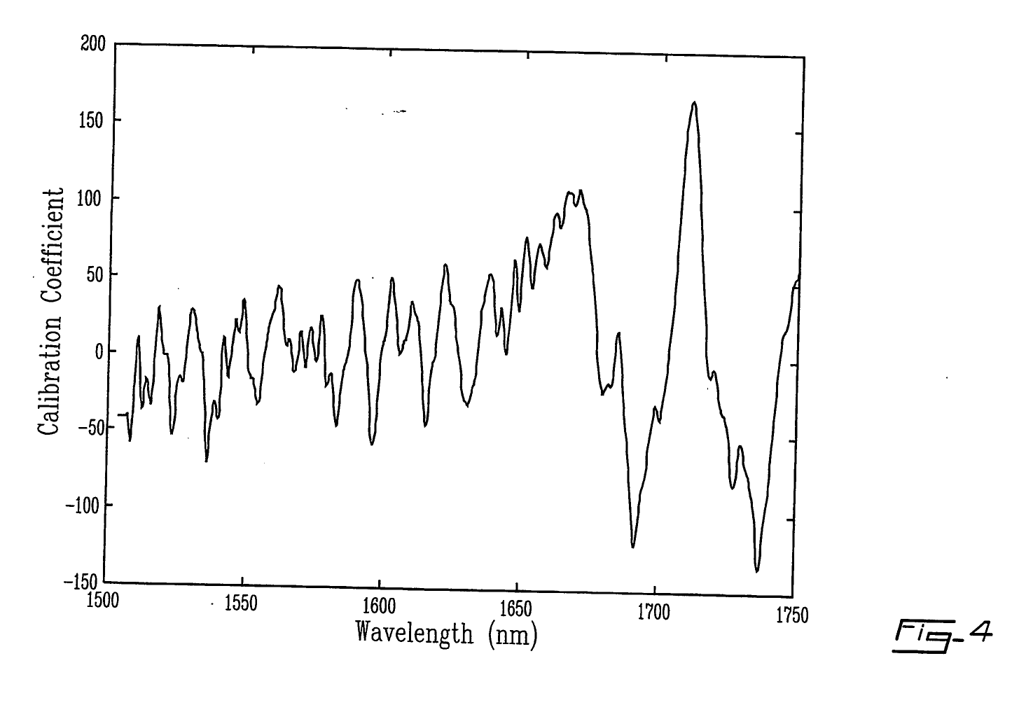

As shown in FIG. 3, the minimum of the prediction error sum of squares (PRESS) plot is reached with 4 factors for lactate. This corresponds to the standard error in the determination of lactate within the 1500 to 1750 nm range. FIG. 4 shows the calibration coefficients plot based on a 4 PLS model. This represents the calibration coefficients at each wavelength, as determined by PLS. Upon viewing FIG. 4, it should be noted that the peaks magnitude are the important features, and both positive and negative values are significant. In the figure, the peaks at 1680 nm (lactate, fingernail), 1690 nm (lactate, glucose), 1710 nm, 1725 nm (lactate, fingernail) and 1740nm (glucose, fingernail) contribute to the greatest extent to the calibration model.

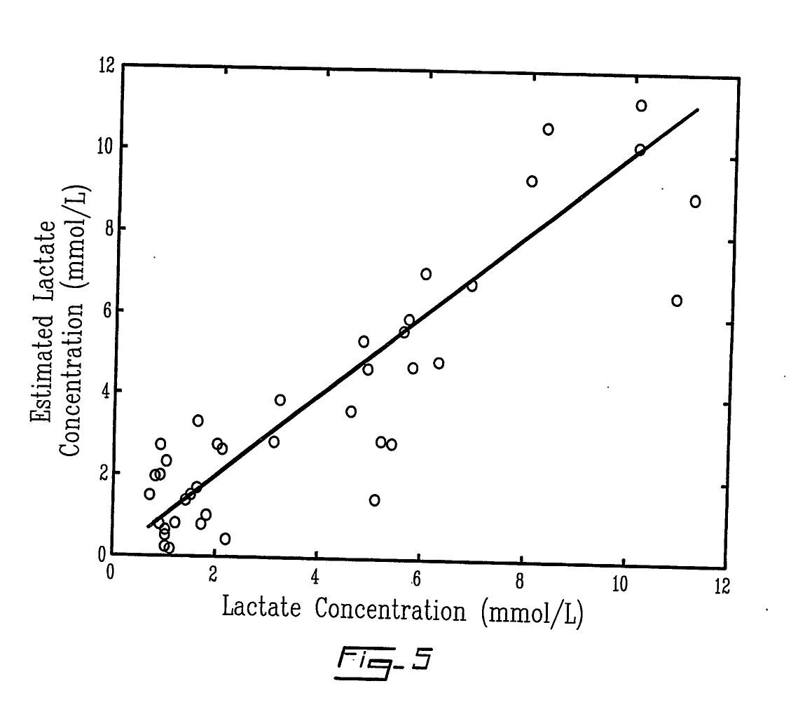

Estimations of lactate concentration in whole blood were obtained by the scalar product of the calibration coefficients vector and each spectrum of the data set. Results using 4 PLS factors are shown in FIG. 5. Correlation between the data and the line of identity, resulted in a correlation coefficient (r) of 0.74. The standard error of cross-validation (SECV) on the linear regression was calculated to be 2.21 mmol/L. The spread seen in the data possibly comes from small variations in blood composition or in the nail bed during exercise within individuals. However, as shown in a previous studies where lactate was measured in human plasma and blood, no particular grouping in the data is seen (Lafrance D. et al., Appl. Spectrosc. 54: 300-304, 2000; Lafrance D. et al., Talanta. (To be published). This consideration indicates that possible variations in blood composition between individuals have little impact on the model. Likewise, FIG. 5 showed that although tight correlation of the data is not apparent, the large change in lactate induced by exercise is easily distinguished. This will also be expected in illness situations.

The PLS model was also used to estimate glucose concentration. The minimum standard error in the determination of glucose was achieved by using thirteen factors. However, after a F-test significance comparison was used to determine the significant number of factors, no difference was found statistically between thirteen and four factors. When four factors are used to build the PLS model for glucose, the correlation coefficient (r) gave 0.37 and the standard error of cross-validation (SECV) on the linear regression was calculated to be 1.53 mmol/L. This result indicates that from the two species, lactate is most likely to be the one that can be monitored using the NIR diffuse reflectance in digits such as fingers.

As mentioned previously, a blood lactate concentration of less than 2 mmol/L is considered as normal (Mizock B. A. et al., Crit. Care Med. 20: 80-93, 1992). Therefore, lactate concentrations changes above 2 mmol/L are particularly important to detect. The current model represents the minimum needed to monitor lactate changes that could occur around that threshold value. Most of the variation appears to come from baseline differences of blood within each of the subjects and the contribution of the fingernail and the fingernail bed to the spectra. To test models with reduced blood composition difference and fingernail contribution, spectra from volunteers at rest were subtracted from the other spectra of each volunteer with the corresponding measured lactate referenced to the standard. This operation is equivalent to a baseline correction for each individual, which is easily accomplished in the clinic.

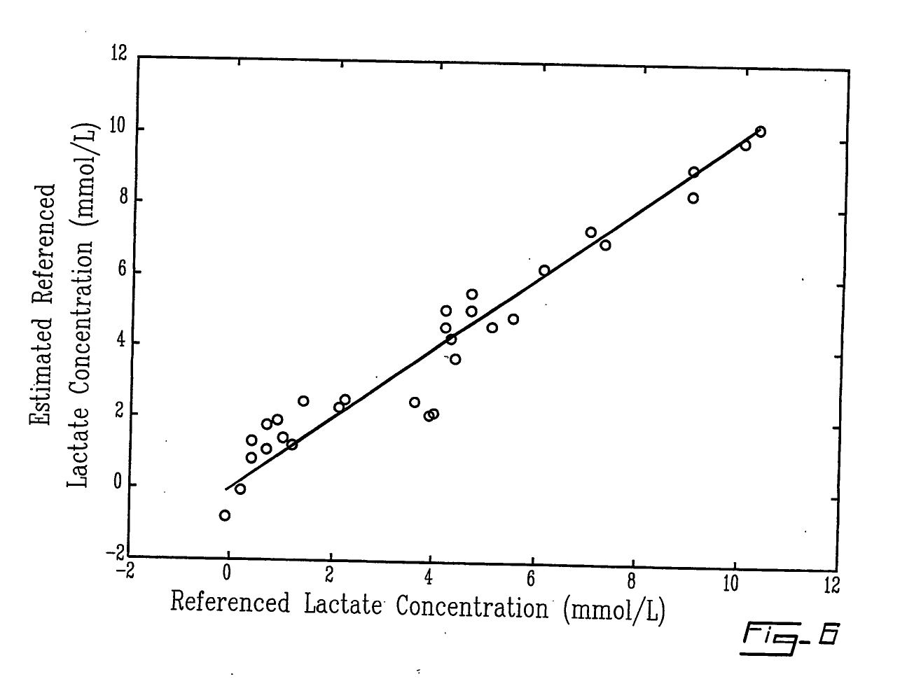

The two pre-processing steps were applied on the resulting spectra and the PLS routine was recalculated. The minimum number of PLS factors to use, calculated with an F-test at a 95% confidence level, was five. FIG. 6 shows the estimations of in vivo referenced lactate concentrations using the 5 PLS factors. Correlation between the data and the line of identity gives a correlation coefficient (r) of 0.97. The standard error of cross-validation (SECV) on the linear regression is 0.76 mmol/L. The standard error has decreased by a factor of three. This translates to a significant improvement in the capability of the model to estimate lactate concentration change. These results indicate the potential of referenced lactate measurements for in vivo physiological or clinical assessment when lactate change in an individual is significant.

It will be appreciated that methods other than PLS can be used to determine the calibration coefficient. For example it may be possible to use empirically determined coefficients that provide a lactate concentration falling in a desired range of concentrations.

In another embodiment of the invention, NIR—Raman spectroscopy may also be used to determine lactate levels in vivo. Thus, NIR light may be injected at one desired wavelength and Raman-shift signals arising from the interaction of the injected light with lactate may be detected at a plurality of wavelengths. The optical signal thus generated may then be analyzed as described above to determine lactate levels.

It will be appreciated that the NIR reflectance data can be acquired at predetermined times. In particular acquisition of data can be synchronized with blood volume variations in the body part where the measurements are taken to account for variations in the optical signal as a result of the normal variations generated by the cardiac cycle. That is to say, variations in localized blood volume arising from variations in the blood flow. These variations may also arise from artificial variations in blood volume in clinical situations such as blood dialysis, surgery or the like.

In a further embodiment of the method of the present invention the optical signal is obtained as a continuous signal over time to generate a “wave” signal pattern reflecting the changes in blood flow. Values of the optical signal can then be extracted at predetermined times within the “wave” cycle. Also, the “wave” optical signals of two or more wavelengths can be compared to provide information on the relative levels of selected blood constituents.

In a further embodiment, levels of lactate can be obtained for the systolic and the diastolic phase of the cardiac cycle to provide a relative optical signal independent of blood volume variations used to calculate lactate levels. Furthermore, it is possible to use the ratio of the “wave” signal resulting from variations in blood volume to that of a steady-state signal (a signal not sensitive to the variation in blood volume) as a way of determining the portion of the signal contributed by blood only. This advantageously provides lactate measurements that are substantially independent of measurement conditions which could affect the reproducibility of the measurements. Such measurement conditions may include but are not limited to the position of the optical coupling means on the body part, intensity of the source and the like.

In a further aspect of the invention there is also provided a system for the in vivo measurement of lactate levels using NIR reflectance spectroscopy. The system comprises a NIR light source 10, means for optically coupling 12 the light source with the body part 14 from which the measurements will be obtained, means for optically coupling 16 the body part 14 with a detector 18, a processor means 20 to process the optical signal exiting the body part and generate a lactate level or concentration and a monitoring means 22 for comparing the measured lactate level with predetermined values of lactate and signaling to a user any difference between the compared values. The processor means of the system may also process the data collected by the detector to determine the wavelengths to be used for the measurements. This determination can be achieved as explained supra using PLS analysis for example. The processor means may be linked to a wavelengths selector 24 to control the wavelengths at which the source will illuminate the body part and the operational wavelengths for the detector. It will be appreciated that the detector can be selectively gated for certain pre-determined wavelengths. Alternatively the wavelength selector may control wavelengths selection means such as filters for example.

The means for optically coupling may be mirrors, lenses, optic fibers and the like. The detector means may be any suitable detector operating in the NIR region of the spectrum.

The system may also comprise a synchronizer means 26 for synchronizing the acquisition of data with a desired event such as the cardiac cycle for example. The synchronizer is preferably linked to the detector, the source and the monitor and any other device that can record the event such as an electrocardiograph for example.

In a further embodiment, lactate levels may also advantageously be measured using NIR transmission spectroscopy using blood samples. In this embodiment a NIR spectrum of a blood sample is obtained. Estimation of lactate concentration is then obtained by the scalar product of predetermined regression calibration coefficients vectors as will be further explained below.

### EXAMPLES

### Example 1

### Sample Collection

Ten healthy adult subjects (six males and four females) were tested during maximal effort made during a 30-s sprint on a modified isokinetic cycle. The cycle was modified to have the pedal speed fixed and effort translated into greater force generation Lands L. C. et al., J. Appl. Physiol. 77: 2506-2510, 1994. The study was approved by the Ethics Committee of the Montreal Children's Hospital, in accordance with the Helsinki Declaration of 1975. After signed informed consent, and prior to exercise, an intravenous line was placed in the antecubital fossa, and kept patent (open) with a 0.9% saline solution. Blood was sampled at four time intervals: (1) just prior to exercise; (2) at the end of exercise; (3) 5 min. following exercise; (4) 10 min. following exercise. This approach was used in an attempt to induce changes within the human physiological ranges for lactate, while minimizing covariance with other species. Blood was drawn into tubes containing lithium heparin beads (Sarstedt Inc., St-Laurent, Quebec) and immediately transferred to pre-chilled 0.75 mL microvette tubes containing 1 mg/mL of sodium fluoride, to arrest glycolysis. Samples were then spun at 15 000 rpm at room temperature for 5 minutes in an Eppendorf microcentrifuge Model 5417C (Eppendorf Scientific, Westbury, N.Y.) to remove plasma for analysis. Plasma samples were each assayed once on a Kodak (Vitros) Model 750 (Orthoclinical Diagnostics, Rochester, N.Y.) for lactate and glucose. Likewise, to monitor the potential impact on light scattering, blood hematocrit was measured for all samples. For the hematocrit measurement, blood samples were placed in capillary tubes. The tubes were loaded into a centrifuge and spun at 13000 rpm for 1 minute. Hematocrit was measured by reading the volume percentage of the red blood cells in the tubes using a micro-capillary reader.

### Example 2

### Data Collection

Spectra were collected with a Nicolet Magna-IR 550 Fourier transform near-infrared (FT-NIR) spectrometer (quartz beamsplitter). The instrument was equipped with stabilized external quartz tungsten halogen source (300 W, Oriel) and an InSb detector. A sample holder, that allowed the finger to rest in front of the light beam, was used to minimize finger movement during exercise and data collection. Two flat mirrors (Edmund Scientific Company, Inc., Barrington, N.J., USA) were used in the sample compartment to bring light to the fingernail and allow diffuse reflectance NIR spectra to be obtained. The spectral range scanned was from 1000 to 2500 nm (11500-4000cm⁻¹). A total of 64 interferogram scans at a spectral resolution of 16 cm⁻¹ were averaged. Single-beam spectra were computed with a Happ-Genzel apodization and Fourier transformation routines available on the system. Background spectra of air were taken every hour. Skin and body temperatures were monitored during data collection with a copper-constantan thermocouple probe and a thermometer (Becton and Dickinson, Mississauga, Ont.) placed respectively in the hand and the mouth of the subject. In this study, the spectral range from 1500-1750 nm was used to do transcutaneous measurements. There were several reasons that motivated the choice of diffuse reflectance spectroscopy of the human nail bed at these wavelengths. First, the fingernail is relatively transparent in this NIR region with absorption near 1660 and 1740 nm (Alam M. K. et al., Appl. Spectrosc. 53: 316-324, 1999). With the low absorption, a significant portion of the reflectance signal that arises comes from the nail bed or deeper, where the tissue is rich in capillary blood vessels (Alam M. K. et al., Appl. Spectrosc. 53: 316-324, 1999). Furthermore, the root-mean-square (rms) noise of the 100% lines computed across the 1500 -1750 nm range using a linear model is 1.38 micro Absorbance Units (μAU). The signal-to-noise ratio (SNR) at 1690 nm is approximately 20, which is sufficient to distinguish species absorption over the background. Finally, several species like lactate or glucose show absorptivities of acceptable magnitude in this spectral range.

In accordance with the present invention, allow absolute measurement of lactate is also contemplated.

In tests performed, the use of a common spectrum for the relative measurements which would make the results absolute measurement of lactate was tested. Second, the use of ratioing to the background (as is done in pulse oximetry) to provide a correction for each individual has been tested. For these measurements, several different wavelength regions were explored.

Method 1: Common Starting Spectrum.

In the case that a relative measurement of lactate is made for each individual, an initial spectrum is acquired to account to varying tissue baselines between individuals. To provide quantitative absolute measurements of lactate, a common starting spectrum for all individuals is used. For this, the initial spectrum for all of nine subjects was averaged together with the average resting lactate level.

Subsequent spectra from the subjects were subtracted from this value and the lactate calculated using the partial least squares method described above. A leave one subject out cross validation was made with the resultant estimations for lactate. Lactate estimation was possible with reasonable accuracy. Though not being quite as precise as the individually referenced measurements, the lactate measurement was suitable for routine monitoring.

Method 2: Ratioing Spectra to Background.

In pulse oximetry, background corrections are achieved by ratioing the spectral signal from pulsatile variation in the tissue. A similar correction was examined for lactate measurements. Spectra were ratioed to the initial spectrum obtained from each individual. Leave one out calibration was then made using the ratioed spectra and a stepwise multilinear regression which chooses the wavelengths to include in the model which best fits the data. Results were very encouraging. Using a similar wavelength range as the previous lactate measurements, seven wavelengths were selected for the model. The wavelengths used were 1642 nm, 1510 nm, 1689 nm, 1708 nm, 1623 nm, 1655 nm, and 1558 nm, in order of contribution from greatest to least. Results are similar to results using partial least squares. The R2 value obtained was 0.9778. Additionally, three other wavelength regions not previously reported were examined. The wavelength range from 2000-2400 nm gave similar though slightly worse estimates of lactate. The choice of wavelengths was 2088 nm, 2111 nm, 2070 nm, 2289 nm, 2325 nm, 2082 nm, and 2400 nm, again in order of contribution from greatest to least. The R2 value obtained was 0.93841. This is probably due to the poor penetration depth of light into tissue in this region. Very good results were also achieved using the wavelength region 1100-1500 nm. This region of the spectra penetrates deeply into tissue and would be practical for a clinical device. The choice of wavelengths was 1468 nm, 1510 nm, 1113 nm, 1239 nm, 1494 nm, 1172 nm, and 1341 nm, in order of contribution from greatest to least. The R2 value obtained was 0.97631. Finally, reasonable estimates were obtained using the wavelength region between 1000-1100 nm. The choice of wavelengths was 1019 nm, 1011 nm, 1024 nm, 1012 nm, 1058 nm, 1086 nm, and 1030 nm, in order of contribution from greatest to least. The R2 value obtained was 0.93789. Though the lactate estimation was not as good at in the 1100-1500 nm region, the shorter wavelength range is accessible to silicon detectors and allows inexpensive devices to be constructed. The plurality of wavelengths may be provided using a plurality of narrowband light sources, such as LEDs, or by using a broadband light source and filters, or by using a tunable source. Wavelength selection may be performed at the source or at the detector, as desired.

It will also be appreciated that the present invention may be applied to measure lactate levels in body fluid in vivo by measurement across the skin or in body cavities, such as orally or vaginally. In a preferred embodiment, the invention may be used in a vaginal probe to measure lactate in amniotic fluid. Using the present invention, the light source and detector can be provided at or optically coupled to the tip of the vaginal probe.

The embodiment(s) of the invention described above is (are) intended to be exemplary only. The scope of the invention is therefore intended to be limited solely by the scope of the appended claims.

1. A method for measuring lactate in vivo comprising:
optically coupling a body part with a light source and a light detector said body part having tissues comprising blood vessels;
injecting near-infrared (NIR) light at a plurality of wavelengths in said body part;
detecting, as a function of blood volume variations in said body part, light exiting said body part at at least said plurality of wavelengths to generate an optical signal;
processing said optical signal as a function of said blood volume variations to obtain a lactate level in blood.

2. A method for measuring lactate in vivo comprising:
optically coupling a body part with a light source and a light detector said body part having tissues comprising blood vessels;
injecting NIR light at one wavelength in said body part;
detecting, as a function of blood volume variations in said body part, light exiting said body part at a plurality of wavelengths to generate an optical signal due to a Raman shift from lactate;
processing said optical signal as a function of said blood volume variations to obtain a lactate level in blood.

3. A method for measuring lactate in vivo comprising:
optically coupling a body part with a light source and a light detector said body part having tissues comprising blood vessels;
injecting near-infrared (NIR) light one or more wavelengths in said body part;
detecting, as a function of blood volume variations in said body part, light exiting said body part at a plurality of wavelengths to generate an optical signal:
processing said optical signal as a function of said blood volume variations to obtain a lactate level in blood, said processing comprising:
a) determining a regression calibration coefficient vector for each of said plurality of wavelengths;
b) obtaining a scalar product from said calibration coefficient vector and an amplitude of each of said plurality of wavelengths.

4. The method as claimed in claim 3, wherein said plurality of wavelengths have an absorption coefficient that is substantially independent of water concentration.

5. The method as claimed in claim 4, wherein said injecting and said detecting is synchronized with changes in blood volume in said body part.

6. The method as claimed in claim 5 wherein said changes in blood volumes are due to cardiac cycle.

7. The method as claimed in claim 6 wherein said lactate level is a relative level between systolic and diastolic parts of said cardiac cycle.

8. The method as claimed in claim 3, wherein said injecting and said detecting produces a time-varying optical signal, said time-varying optical signal being a function of changes of blood volume in said body part.

9. The method as claimed in claim 8, wherein said changes in blood volumes are due to cardiac cycles.

10. The method as claimed in claim 9, wherein said detecting comprises detecting light at said plurality of wavelengths to generate said time-varying optical signals and a steady state signal and wherein a ratio of said time varying optical signals and said steady state signal is obtained to thereby producing a relative signal substantially reflecting said lactate level in blood.

11. The method as claimed in claim 3, wherein said body part is a digit comprising a nail and a nail bed.

12. The method as claimed in claim 11, wherein said NIR light is injected through said nail.

13. The method as claimed in claim 12, wherein said exiting light is detected though said nail bed.

14. The method as claimed in claim 13, wherein said illuminating comprises:
a) immobilizing said digit in a sample compartment; and
b) directing said NIR light on said nail.

15. The method as claimed in claim 14, wherein said plurality of wavelengths is at least four.

16. The method as claimed in claim 15, wherein said light detected is at a same wavelength as said light injected, and the wavelengths are 1680 nm, 1690 nm, 1710 nm and 1725 nm.

17. The method as claimed in claim 16, wherein a reference optical signal is subtracted from said optical signal.

18. The method as claimed in claim 3, further comprising:
activating an alarm when said lactate level differs from a predetermined level indicative of an abnormal lactate-dependent condition; and
taking at least one corrective action in response to said abnormal lactate-dependent condition.

19. The method as claimed in claim 18, wherein said abnormal lactate-dependent condition is high lactate level in an exercising subject and wherein said corrective action comprises stopping said subject from exercising.

20. The method as claimed in claim 18, wherein said abnormal lactate-dependent condition is a clinical condition in a subject selected from myocardial infarction, cardiac arrest, circulatory failure, emergency trauma.

21. A system for measuring in vivo lactate levels comprising:
a NIR light source;
a source coupler optically coupling said light source to a body part;
detector coupler optically coupling said body part to a detector for measuring light exiting 'said body part and producing an optical signal;
processor receiving said optical signal and generating a measured lactate level value; and
a monitoring device comparing said measured lactate level value with at least one predetermined lactate value, and
triggering a signal perceptible by a user when said compared values are within a predetermined range.

22. The system as claimed in claim 21, wherein said processor determines said predetermined wavelengths.

23. The system as claimed in claim 22, further comprising a wavelengths selector selecting said source wavelengths and said detector operating wavelengths.

24. The system as claimed in claim 23, further comprising a synchronizer synchronizing said measuring with a desired event.

25. The system as claimed in claim 24, wherein said event is cardiac cycle.

26. The system as claimed in claim 25, wherein said synchronizer is operationally coupled to an electrocardiograph.
